(19) United States
(12) Patent Application Publication
(10) Pub. No.: US 2010/0038374 A1
(43) Pub. Date: Feb. 18, 2010

(54) Waste-Reducing Dispenser for Dispending Bandages and/or Transdermal Delivery Devices and Related Methods

(76) Inventor: Judith Campi, Paulsboro, NJ (US)

Correspondence Address: FLASTER/GREENBERG P.C.;Four Penn Center, 1600 John F. Kennedy Boulevard, 2nd Floor, PHILADELPHIA, PA, 19103, US

(21) Appl. No.: 12/542,396

(22) Filed: Aug. 17, 2009

Related U.S. Application Data
(60) Provisional application No. 61/189,260, filed on Aug. 15, 2008.

(51) Int. Cl. B65D 83/00; B26D 7/27; A61F 13/00

(52) U.S. Cl. 221/46; 53/396

(57) ABSTRACT

The invention provides a device for dispensing bandages from a continuous strip comprising: (i) housing having a first and a second side wall and an aperture; (ii) at least one post extending inwardly from at least one side wall, (iii) a continuous strip of bandage material having a first terminus and a second terminus, wherein the continuous strip of bandage material is disposed within the housing and at least the first terminus is threaded through the aperture. The device may be used to dispense bandages or adhesive transdermal delivery device, such as “patches”.

### CROSS REFERENCE TO RELATED APPLICATION

This application claims priority under 35 U.S.C. §119(e) to U.S. Provisional Patent Application No. 61/189,260, filed Aug. 15, 2008, the entire disclosure of which is incorporated herein by reference.

### BACKGROUND OF THE INVENTION

The healing of minor wounds and abrasions to the skin's surface, such as cuts and scrapes, is often promoted or facilitated by the use of protective bandages, which protect the wound from further damage and prevent exposure of the wound to eternal contaminants which may infect of further aggravate the wound. In addition, as any parent of small children will attest, the placebo effect of immediate application of a protective bandage to a wound, however minor, is not to be underestimated.

Conventional adhesive bandages, such as those sold under the BAND-AID trademark are individually packaged. Use of one bandage generates at least four pieces of trash (2 sides of outer covering and 2 parts of adhesive backing). Moreover, the individually package conventional bandages are not packed for portability—the integrity of the papers wrappers is easily breached within a typical travel or portable environment (such as a brief case, pocket or handbag) and, consequently, the sterility or cleanliness of the bandage is compromised.

There exists a need in the art for adhesive bandages that may be consumed with reduced waste, yet remain sterile and uncompromised in transit or in the medicine cabinet.

### BRIEF SUMMARY OF THE INVENTION

The invention provides a device for dispensing bandages from a continuous strip comprising: (i) housing having a first and a second side wall and an aperture; (ii) at least one post extending inwardly from at least one side wall, (iii) a continuous strip of bandage material having a first terminus and a second terminus, wherein the continuous strip of bandage material is disposed within the housing and at least the first terminus is threaded through the aperture. The device may be used to dispense bandages or adhesive transdermal delivery device, such as “patches”. Also provided are methods of packaging reduced waste bandages that include providing a continuous strip of bandage material having a first terminus and a second terminus, providing a housing having a first and a second side wall, an aperture and at least one post extending inwardly from at least one side wall, and engaging the continuous strip of bandage material with the at least one post and threading at least the first terminus the aperture.

### BRIEF DESCRIPTION OF THE SEVERAL VIEWS OF THE DRAWINGS

The foregoing summary, as well as the following detailed description of several embodiments of the invention, will be better understood when read in conjunction with the appended drawings. It should be understood that the invention is not limited to the precise arrangements and instrumentalities shown. In the drawings:

FIG. 1 is a perspective view of an embodiment of the device of the invention;

FIG. 2 is a cross-sectional view of the device of FIG. 1;

FIG. 3 is a cross sectional view of a second embodiment of the invention;

FIG. 4 is a from view of the embodiment of FIG. 3, showing the aperture; and

### DETAILED DESCRIPTION OF THE INVENTION

The invention includes the device for dispensing bandages from a continuous strip and related methods. The device is advantageous because it permits the dispensing of adhesive bandages and/or adhesive transdermal drug delivery patches (collectively referenced herein as “bandages”), similar, for example, to those sold under the Band Aid trademark, but reduces any trash or waste associated with those bandages. In addition, because the bandage is packaged within the waste-reducing dispenser, it can be carried from place to place in a pocket, handbag, briefcase, or medicine kit without any loss of sterility or other damage from, for example, liquids or dust.

The device includes a housing that has at least a first sidewall and a second sidewall. The housing may be of any shape or configuration, such as a rectangle, square, sphere, or flattened sphere. If desired, the housing may be of a novelty shape, such as a seashell, an apple, a baseball or football or other sport equipment, a cat, a dog, a teddy bear, other animals and the like.

Materials for use in making the housing may be any known or to be developed in the art and can include, for example, cardboard, metal, plastics, fiberglass, and paper, preferably paper laminated with plastic film. It may be preferred that the material is substantially impermeable to microorganisms, liquids and dust. In an embodiment, it may be preferred that the material chosen for the housing sufficiently durable to maintain structural integrity after of at least one sterilization process (such as autoclaving, radiation, heat, chemical or steam treatments or other medically accepted processes that renders the material sterile).

The housing may be formed as a unitary structure or it may be composed of several parts affixed together. If the housing is not unitary, any seams or joins may preferably be sealed, to render seams/joins substantially impermeable to microorganisms, liquids and dust.

For example, the housing may be made of a thermoplastic and in the shape of a flattened sphere that is blow molded so that no seams or joins are present. In another embodiment the housing may be made of a durable cardboard that is glued at the seams and coated in a plastic coating to seal seams and to provide additional durability.

The housing should contain an aperture through which the at least the first terminus of the continuous strip may be threaded. The aperture may be any configuration or geometry, such as circle, square, rectangle, half-moon, etc. The aperture may be located in one of the sidewalls. Alternately, the housing may include an elongated extension or conduit at the end of which is situated the aperture.

If more than one aperture is present, they may be disposed in one or both of the sidewalls or, if a third and fourth sidewall are present, in any of these.

The aperture contains at least an outer edge. The outer edge may further comprise a cutting edge, such as a serrated cutting edge, a straight cutting edge, or a pronged cutting edge. The cutting edge may be attached to the outer edge of the aperture as a separate and distinct part or the cutting edge may be formed unitarily with the sidewall, such as, for example, if the sidewall is molded of a thermoplastic the mold can be modified to include a cutting edge at the aperture.

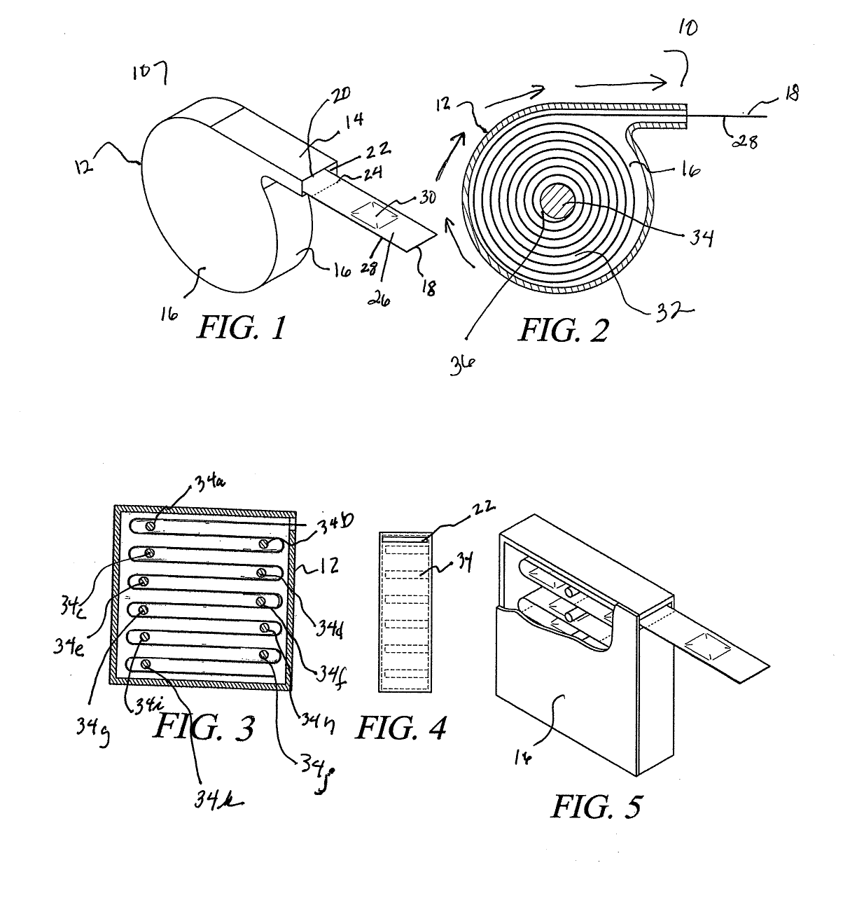

At least one of the sidewalls comprises a post that extends inwardly into the cavity of the housing. The post may be any configuration, such as, for example, a rod, a bar or a spindle type post, and can have any cross section such as a circle or a square or a rectangle. One or more posts may be present depending on how the continuous strip of bandage material is to be disposed within the housing. For example, if the continuous strip of bandage material is configured into a roll, such as, for example, that shown in e.g., FIG. 1, only one post may be necessary. Alternatively, the continuous strip of bandage material may be configured in a zigzag format in the interior of, for example, if a housing that is in the form of a rectangular prism one or more posts may be useful as is illustrated in FIG. 3.

The housing may include a cover that seals the aperture when the bandage is not being dispensed. The cover may be a hinge cover that is attached to the housing, e.g., a door like structure that is attached to an outer edge of the aperture and can be flipped open and shut. The lid can also include a removable and replaceable adhesive panel, or a self-sealing rubber flap, or a removable cap.

Also within the device is a continuous strip of bandage material. As used herein, the term “bandage material” includes adhesive bandages for use in the treatment and protection of wounds, abrasions, burns, and broken skin, and/or adhesive transdermal drug delivery patches, which are applied to unbroken skin for transdermal drug delivery (systemic or local).

The continuous strip may, however, contain perforations or other breaks in the material to permit dispensing of a single bandage or drug dosage, so long as the perforation or breaks are not significant enough to fully separate individual portions of the strip. It may be preferred that the continuous strip has an X axis that is longer than the Y axis. Preferably, any perforations or breaks will be in the direction of the Y axis of the strip.

In an embodiment, it may be preferred that the continuous strip includes at least a first layer and a second layer. The first layer may include a skin or wound contact material (hereinafter collectively “wound contact material”) for coming in contact with the wound, burn, abrasion, or other break in the skin. The wound contact material may be any known or to be developed in the art, including, for example, a woven textile, such as a gauze, oppressed fiber textile, such as a felt, a gel polymer, such as those used in the treatment of burns to keep them moist.

The wound contact material may be fabricated from any materials known in the art such as natural materials (like cotton, silk, paper or wood fibers) or synthetic, such as nylons and other synthetic fibers. The wound contact material may be treated with, impregnated with, or have woven into it, substances that may aid the healing of a wound either by preventing infection, inflammation or other means. For example, the wound contact material may comprise a pharmaceutical material, an anti-inflammatory agent, an antiseptic agent, an antifungal agent, a vitamin, and an antioxidant.

In one embodiment, the continuous bandage material is not applied to wounds or open skin, but to unbroken skin in order to deliver a pharmaceutical transdermally. In such cases, the first layer, second layer, or both, may be impregnated with the transdermal pharmaceutical, such as, for example, fludroxycortide, nicotine, scopolamine, estrogen, and other hormones, nitroglycerin, diclosenac, etolamine, and clonidine.

In an embodiment, the second layer includes an adhesive, such as a pressure sensitive adhesive, a contact adhesive, and a reusable adhesive. The adhesive may be any known or to be developed in the art, such as, for example, an epoxy resin, a meleamaic acid-based material, an acrylate polymer or copolymer and/or a formaldehyde resin. Other adhesives may be preferred, depending on the treatment to be delivered. Although any resins known or to be developed in the art may be used, it may be preferred that specific resins include dodecyl maleamaic acid and octadecyl maleamaic acid, tetrahydrosurfuryl acrylate, and tertbutylphenol formaldehyde resin.

In an embodiment, the continuous strip of bandage further comprises a third layer that is a backing material. Backing material may be of any known or to be developed in the art. It can include fabrics, other textiles, plastic sheets or films, fiberglass and paper.

In an embodiment, the backing material is transparent or translucent, so that the natural pigmentation of the skin of the wearer shows through, making detection of the bandage or delivery device less apparent.

In embodiment, the continuous strip of bandage material comprises a first layer, a second layer, and a third layer, as described above, wherein the second layer and the third layer are each dimensionally similar, i.e., they have the same size and shape so that when placed on top of one another there is minimal overlap. Additionally, the first layer has a dimension that is less than the dimensions of the third and the second layers, permitting the adhesive layer (second layer) to overlap at least some portion of the first layer, allowing for adherence to the skin's surface.

Also included within the scope of the invention are methods of packaging reduced waste bandages. The methods include providing a continuous strip of bandage material and a housing that has at least a first and a second sidewall as well as an aperture. In the practice of the method, the continuous strip of bandage material is positioned within the housing and engaged with the at least one post, such that it can be threaded through the aperture, at least at the first terminus. In the method of packaging, the housing is sealed to maintain a substantially sterile interior, thereby preventing the introduction of dust and/or microbes to the skin's surface upon application of the bandage.

FIG. 1 shows a perspective view of an embodiment of the waste-reducing dispenser (10), wherein the housing (12) comprises a flattened sphere-like shape with a protruding dispensing conduit (14). In FIG. 1 only one sidewall (16) is visible. The continuous strip of bandage material (18) is threaded through the aperture and can be broken off against the outer edge (20) of the aperture, top or bottom, (22) at the perforations (24). In the embodiment of FIG. 1 the continuous strip (18) is shown having a transparent backing layer (26) and adhesive layer (28). The wound/skin contact material (30) is shown being not a format commensurate with the format of the backing and adhesive layers.

FIG. 2 shows a cross section of the dispenser (10) of FIG. 1. As can be seen from the cross section, the continuous strip of bandage material (18) is configured into a roll with the adhesive layer (28) of the continuous strip facing the interior of the roll. The housing (32) contains one post (34) that extends from the sidewall (16) and the central axis portion of the roll (36) engages the post (34). When the continuous strip of bandage material is pulled through the aperture (22) to dispense, the tension force is exerted along the continuous strip against the spindle or post (34).

FIG. 3 shows a side view in cross section of a second embodiment of the invention, wherein the housing (12) is in the form of a rectangular prism. As can be seen from FIG. 3, the continuous strip of bandage material (18) is disposed within the housing in an accordion-like fashion. The folds may be held in place or guided through the housing by the post(s) (34a-k). However, in an embodiment, it is preferred that only the upper portion of the housing contains the posts (for example, only those posts labeled 34a-d are present), so that the greater length of bandage material may be fit within the housing (the strip may be folded and slightly compressed in the bottom portion of the housing. FIG. 4 and FIG. 5 show a front view and a perspective cut away view respectively of a similar embodiment and illustrates an aperture that is flush with the sidewall and a dispenser with the posts (34) situated more centrally.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A device for dispensing bandages from a continuous strip comprising:
a. housing having a first and a second side wall and an aperture;
b. at least one post extending inwardly from at least one side wall,
c. a continuous strip of bandage material having a first terminus and a second terminus,
wherein the continuous strip of bandage material is disposed within the housing and at least the first terminus is threaded through the aperture.

2. The device of claim 1, wherein the housing further comprises at least one post that projects from a sidewall, wherein the continuous strip of bandage material is configured in contact with the post such that a tension force is exerted along the continuous strip when the first terminus is pulled through the aperture.

3. The device of claim 1, wherein the continuous strip is configured into a roll having a central axis portion that engages with the post.

4. The device of claim 2, wherein the housing comprises about 3 to about 6 posts.

5. The device of claim 1, wherein at least a portion of an outer edge of the aperture comprises a cutting edge.

6. The devices of claim 5, wherein the cutting edge is a serrated edge.

7. The device of claim 1, wherein the continuous strip of bandage comprises at least a first layer that includes a wound contact material and a second layer that includes an adhesive.

8. The device of claim 7, wherein the wound contact material is chosen from a woven textile, a pressed fiber textile, a gel polymer, a paper, wood fiber textile, bacterio-static gauze and cotton gauze.

9. The device of claim 7, wherein the wound contact material further comprises a pharmaceutical material, and antiseptic, and an antifungal agent,

10. The device of claim 7, wherein the adhesive is chosen from a pressure-sensitive adhesive and a contact adhesive.

11. The device of claim 7, wherein the adhesive comprise a material chosen from an epoxy resin, an maleamic acid -based material, an acrylate polymer and a formaldehyde resin.

12. The device of claim 1, wherein the continuous strip of bandage further comprises a third layer of backing material.

13. The device of claim 1 wherein the backing material is chosen from a plastic, a polymer, and a textile.

14. The device of claim 1, wherein the continuous strip of bandage material comprises:
a. first layer that includes a wound contact material;
b. a second layer that includes an adhesive; and
c. a third layer that comprises a backing material,
wherein the second layer and the third layer are each have a dimension that is substantially similar to one another and the first layer has dimension that is less than the dimension(s) of the third and the second layer.

15. The device of claim 1, wherein the continuous strip of bandage is perforated along an Y axis of the strip at an increment.

16. The device of claim 15 wherein the increment is about 5 cm to about 10 cm in length.

17. The device of claim 15 wherein the increment is about 8 cm in length.

18. A method of packaging reduced waste bandages comprising:
a. providing a continuous strip of bandage material having a first terminus and a second terminus,
b. providing a housing having a first and a second side wall, an aperture and at least one post extending inwardly from at least one side wall, and
c. engaging the continuous strip of bandage material with the at least one post and threading at least the first terminus the aperture.

19. The method of claim 18, wherein the housing is sealed to maintain a substantially sterile interior.

20. The method of claim 18, wherein the continuous strip of bandage material comprises:
a. first layer that includes a wound contact material;
b. a second layer that includes an adhesive; and
c. a third layer that comprises a backing material,
wherein the second layer and the third layer are each have a dimension that is substantially similar to one another and the first layer has dimension that is less than the dimension(s) of the third and the second layer.
